(19) United States
(12) Patent Application Publication
(10) Pub. No.: US 2003/0158439 A1
(43) Pub. Date: Aug. 21, 2003

(54) Preparation of alkynecarboxylic acids and alkyne alcohol esters of alkynecarboxylic acids by oxidation of alkyne alcohols

(75) Inventors: Jurgen Stohrer, Pullach (DE); Elke Fritz-Langhals, Ottobrunn (DE); Christian Bruninghaus, Unterhaching (DE); Dagmar Stauch, Munchen (DE)

Correspondence Address: COLLARD & ROE, P.C., 1077 Northern Boulevard, Roslyn, NY, 11576, US

(73) Assignee: CONSORTIUM FUR ELEKTROCHEMISCHE INDUSTRIE GMBH

(21) Appl. No.: 10/365,887

(22) Filed: Feb. 13, 2003

(30) Foreign Application Priority Data
Feb. 15, 2002 (DE) 102 06 619.1

(51) Int. Cl. C07C 51/295

(52) U.S. Cl. 562/539

(57) ABSTRACT

A process for preparing alkynecarboxylic acids and alkyne alcohol esters of alkynecarboxylic acids, includes an oxidation reaction of an alkyne alcohol with from 1 to 10 molar equivalents of a hypohalite based on the number of functional groups to be oxidized in the presence of a nitroxyl compound at a pH of less than 7. There is also a partial oxidation reaction of the alkyne alcohol with from 0.5 to 5 molar equivalents of a hypohalite based on the number of functional groups to be oxidized in the presence of a nitroxyl compound at a pH of less than 7.

### BACKGROUND OF THE INVENTION

1. Field of the Invention

The present invention relates to processes for oxidizing alkyne alcohols (alkynols) to alkynecarboxylic acids (alkynoic acids) and also to the preparation of alkyne alcohol esters of alkynecarboxylic acids.

2. The Prior Art

Alkynoic acids are important synthetic building blocks. Of particular importance are propiolic acid and acetylenedicarboxylic acid which are used to build rings in cycloadditions, in particular Diels-Alder reactions and 1,3-dipolar cycloadditions, and in nucleophilic addition reactions (overview in Ullmann's Encyclopedia, 6th Edition, 2001 electronic release; “Carboxylic acids, aliphatic 5.2”).

The oxidation of alkynols to alkynoic acids has been described in the prior art (overviews in Ullmann's Encyclopedia, 6th Edition, 2001 electronic release; “Carboxylic acids, aliphatic 5.2”; Houben-Weyl volume V/2a, 4th edition 1977, “Alkynes”).

For example, propiolic acid is obtained by anodic oxidation of propargyl alcohol (Wolf, Chem. Ber. 1954, 87, 668). Acetylenedicarboxylic acid is likewise obtained by anodic oxidation of 2-butyne-1,4-diol. However, the electrochemical process has the disadvantage of the use of lead dioxide anodes. This leads to the contamination of the electrolytes with lead ions and can generally only be used in production at high capital cost. In addition, the decarboxylation of the product proceeding as a side reaction leads to technically undesired formation of large amounts of CO₂ and acetylene which have to be disposed of. Also, the yields in the case of propiolic acid are relatively low (less than 50%).

The analogous anodic oxidation on nickel oxide anodes (Kaulen and Schäfer, Tetrahedron 1982, 38, 3299) requires low current densities and very large electrode surface areas, which leads to a further increase in the capital costs. In addition, the activated nickel surface is passivated during the electrolysis and frequently has to be regenerated which leads to an increase in the process costs.

Propiolic acid can also be obtained by oxidation of propargyl alcohol with Cr(VI) oxide in sulfuric acid. Good yields can be achieved, but large amounts of toxic and environmentally hazardous heavy metal salts have to be disposed of. The analogous reaction of 2-butyne-1,4-diol leads to only a 23% yield of acetylenedicarboxylic acid (Heilbron, Jones and Sondheimer, J. Chem. Soc. 1949, 606).

A known nonoxidative preparation process of propiolic acid and acetylenedicarboxylic acid is the reaction of metal acetylides with CO₂. However, this requires the use of expensive metal bases and, owing to the use of acetylene, is technically not without risk. The yields of this process in the case of propiolic acid are likewise only 50%.

In a further process for preparing acetylenedicarboxylic acid, fumaric acid is initially converted with bromine to meso-dibromosuccinic acid, which is then isolated and dehalogenated in a further stage. This two-stage process is time-consuming and laborious (T. W. Abbott et al., Org. Synth. Coll. Vol. II, 1943, 10).

The prior art discloses general oxidation processes of alcohols to carboxylic acids with the aid of nitroxyl compounds as catalysts, in particular with the aid of TEMPO (2,2,6,6-tetramethylpiperidin-1-oxyl) and its derivatives (overview in A. E. J. de Nooy, A. C. Besemer and H. V. Bekkum, Synthesis 1996, 1153).

Numerous TEMPO derivatives have been described as oxidation catalysts, including TEMPO derivatives on polymeric supports, for example PIPO (polyamine-immobilized piperidinyl oxyl) (Dijksman et al., Synlett 2001, 102).

These TEMPO-catalyzed oxidations are carried out in biphasic systems, for example methylene chloride/water (P. L. Anelli, C. Biffi, F. Montanari and S. Quici, J. Org. Chem. 1987, 52, 2559).

In all of these processes, carboxylic acids may be obtained from alcohols with the additional use of phase transfer catalysts (G. Grigoropoeulou et al., Chem. Commun. 2001, 547-548, P. L. Anelli, C. Biffi, F. Montanari and S. Quici, J. Org. Chem. 1987, 52, 2559). The stoichiometric oxidant used is predominantly bleaching liquor (hypochlorite solution).

In the customary performance of these syntheses in biphasic systems, the oxidant dissolved in the aqueous phase is added in a batch process within a pH range of 9-10 to the organic phase which comprises the alcohol to be oxidized and the nitroxyl compound.

However, no process which can be carried out on the industrial scale for oxidizing unsaturated alkyne alcohols (alkynols), in particular those having terminal acetylene groups, to the corresponding alkynecarboxylic acids (alkynoic acids) using hypochlorite in the presence of nitroxyl compounds has yet been described.

A possible cause is the sensitivity disclosed by the literature of terminal alkyne groups toward bleaching liquor. The CH groups of terminal alkynes are easily converted, for example, to chloroalkynes by bleaching liquor, which are particularly labile in alkaline media and tend to decompose (Straus et al., Ber. Dtsch. Chem. Ges. 1930, 1868).

The prior art discloses that oxidation processes using bleaching liquor and nitroxyl compounds are generally to be considered as unsuitable for the oxidation of unsaturated alcohols (on this subject, compare in particular P. L. Anelli, C. Biffi, F. Montanari and S. Quici, J. Org. Chem. 1987, 52, 2559; P. L. Anelli, F. Montanari and S. Quici, Org. Synth., 1990, 69, 212).

For instance, the reaction of an alkyne alcohol having a nonterminal acetylene group with bleaching liquor and TEMPO by a prior art process delivers only from 5 to a maximum of 20% of the alkynoic acid. The oxidation succeeds only with the use of sodium chloride as oxidant with the addition of catalytic amounts of hypochlorite and TEMPO in a biphasic mixture buffered with phosphate buffer at pH 6-7 (M. Zhao et al., J. Org. Chem. 1999, 64, 2564; WO 99/52849). In this method, the use in particular of the relatively expensive, and moreover explosive, sodium chlorite is disadvantageous. In addition, large amounts of phosphate buffer are required, whose disposal is costly and inconvenient.

### SUMMARY OF THE INVENTION

It is an object of the present invention to provide a process for preparing alkynoic acids and alkyne alcohol esters of alkynecarboxylic acids by the oxidation of alkyne alcohols which avoids the disadvantages known from the prior art.

The present invention provides a process for preparing alkynecarboxylic acids, characterized by the oxidation of an alkyne alcohol with from 1 to 10 molar equivalents of a hypohalite based on the number of functional groups to be oxidized in the presence of a nitroxyl compound at a pH of less than 7.

The invention further provides a process for preparing alkyne alcohol esters of alkynecarboxylic acids, characterized by the partial oxidation of the alkyne alcohol to alkyne alcohol esters of alkynecarboxylic acids with from 0.5 to 5 molar equivalents of a hypohalite based on the number of functional groups to be oxidized in the presence of a nitroxyl compound at a pH of less than 7.

The process product is the ester of the corresponding alkynecarboxylic acid and the unconverted portion of the alkyne alcohol.

For example, when propargyl alcohol is used as the reactant, propargyl propiolate is formed.

In general, the components in the processes according to the invention which are involved in the reaction may be reacted in one phase or distributed over more than one phase.

In possible embodiments of the processes according to the invention, an aqueous phase is used or the processes are carried out in an aqueous phase.

In a further possible embodiment, the processes according to the invention are carried out in a phase which comprises water and one or more water-miscible solvents (cosolvents).

In one embodiment of the monophasic reaction, the alkynol to be oxidized is initially charged in pure form or diluted with water or with one or more inert water-miscible solvents together with the nitroxyl compound as reaction component 1 and admixed with the oxidant as reaction component 2.

In an alternative embodiment, both reaction components are metered in parallel, and the inert water-miscible solvent or the water-miscible solvents or the nitroxyl compound may additionally be initially charged.

The inert water-miscible solvents are preferably selected from the group of ethers, in particular THF and 1,4-dioxane, the nitrites, in particular acetonitrile and DMF, DMSO and tert-butanol.

The alkyne alcohol to be oxidized may be used in concentrations of from 0.1 to 100% by weight, preferably from 20 to 100% by weight, based on the water used for dilution or the water-miscible solvent or the water-miscible solvents.

In a further possible embodiment of the processes according to the invention, the reactions are carried out in multiphasic systems.

Preference is given to using at least one aqueous phase and one organic phase.

In a particularly preferred embodiment, the alkyne alcohol is used optionally in pure form or dissolved in one or more water-immiscible solvents.

When multiphasic operation is effected, the organic phase consists of alkyne alcohol, the nitroxyl compound and, if appropriate, one or more organic solvents. The second aqueous phase comprises the hypohalite. The phase separation may also be caused by the use of a water-immiscible alkyne alcohol as the reactant or by the formation of a water-immiscible alkynecarboxylic acid or alkyne alcohol of an alkynecarboxylic acid as products.

Preferred organic solvents for carrying out the process in a multiphasic system are one or more solvents selected from the group of ethers, in particular THF, methyl t-butyl ether and diethyl ether, acetonitrile, methylene chloride, ethyl acetate, dimethyl sulfoxide (DMSO), tert-butanol and toluene.

The alcohol to be oxidized may be used in concentrations of from 0.1 to 100% by weight based on the organic phase, preferably from 20 to 100% by weight.

In a preferred embodiment of the process for preparing alkyne alcohol esters of alkynecarboxylic acids, this oxidation is carried out in biphasic reaction mixtures using one or more water-immiscible solvents, so that the ester is obtained in the organic phase and can therefore be easily removed. The second aqueous phase comprises the hypohalite. The ester formed may be converted to the corresponding alkynoic acids by treatment with aqueous liquors, and the coproduced alkynol can be recovered and can be used for further oxidations. Alternatively, the esters may also be converted to other alkynoic esters by transesterification.

The advantages of the processes according to the invention are the provision of technically simple processes for oxidizing alkyne alcohols to alkynecarboxylic acids and for preparing alkyne alcohols of alkynecarboxylic acids which use inexpensive hypohalites and remedy the problems known from the prior art.

The processes according to the invention allow even highly water-soluble alkynols and alkynols having terminal alkyne groups to be oxidized to the corresponding alkynecarboxylic acids in a technically simple manner and good yield using inexpensive bleaching liquor (sodium hypochlorite) which is technically simple to use.

The obligatory use of the biphasic systems known from the prior art for the oxidation is no longer necessary, in particular for such substrates.

Especially for substrates having terminal alkyne groups, there were hitherto no reactions provided by the oxidative processes of the prior art which could be realized on the industrial scale and were economically interesting.

Furthermore, the processes according to the invention can be applied to a wide variety of substrates having terminal and nonterminal alkyne groups.

For example, propiolic acid may be obtained from propargyl alcohol by the processes according to the invention in a 45-75% yield, and acetylenedicarboxylic acid from butynediol in a 50% yield.

The wastewater resulting from the reaction comprises only salts which are easy to dispose of such as NaCl and, in contrast to processes in which phosphate buffers have to be used, can be easily disposed of.

In one possible embodiment of the processes according to the invention, the phases taking part in the reaction may also be reacted with each other continuously.

In this case, the reaction components 1 and 2 are fed continuously and, at the same time, the reaction solution formed is continuously withdrawn. The pH may be maintained in the range favorable for the reaction by a third continuous metered addition of bases or acids to the reaction mixture, so that there is a constant pH<7 in the reaction mixture.

The continuous reactors suitable for a continuous process are known to those skilled in the art. An example of an overview of the most important embodiments can be found in “Ullmann's Encyclopedia of Industrial Chemistry”, Vol. B4.

Preferred embodiments for a process carried out continuously are continuously operated tubular reactors, continuously operated loop reactors, continuously operated stirred tanks or stirred tank batteries or a process carried out with the aid of circulation pumps.

When the process described is carried out as a continuous process, an additional advantage is an efficient heat removal from the strongly exothermic reaction process.

The oxidative processes known from the prior art were only possible in batch operation and therefore of little interest with regard to the possibility of realization on the industrial scale and economic viability.

In general, the alkyne alcohols (alkynols) to be oxidized are compounds which contain at least one monovalent group of the formula —CH₂—OH and at least one divalent group of the formula —C≡C—.

The alkyne alcohols to be oxidized are preferably linear or branched primary alcohols having 3-30 carbon atoms, cyclic alcohols having 8-30 carbon atoms or alcohols which are substituted by an aromatic radical and have 6-30 carbon atoms, each of which contains a group of the formula —C≡C—,

where one hydrogen or more than one hydrogen may be independently replaced by F, Cl, Br, I, NO₂, ONO, CN, NC or SCN,

or where one CH₂ group or more than one CH₂ group may be independently replaced by O, NH, C═O, CO₂, S, S═O, SO₂, P═O or PO₂,

or one CH group or more than one CH group may be independently replaced by N, B or P, or quaternary carbon atoms may be replaced by Si, Sn or Pb.

Particular preference is given to the alkyne alcohols R¹—C≡C—CH₂OH, R¹—C≡C—CH₂—CH₂OH or R¹C≡C—CH₂—CH₂—CH₂OH, R¹—O—CR²R³—C≡C—CH₂OH, R¹O—CR²R³—C≡C—CH₂—CH₂OH or R¹—O—CR²R³—C≡C—CH₂—CH₂—CH₂OH

where R¹ is H, methyl, ethyl or a linear or branched C₃-C₁₂ radical, in particular an n-propyl, isopropyl, 1- or 2-n-butyl, 2-methylpropyl, 1-, 2- or 3-n-pentyl, 2- or 3-methyl-1-butyl, 1,2-dimethylpropyl, tert-butyl, neopentyl or tert-pentyl radical,

or a saturated or unsaturated cyclic C₃-C₁₂ radical, in particular a cyclopropyl, cyclobutyl, cyclopentyl, cyclopentenyl, methylcyclopentyl, methylcyclopentenyl, cyclohexyl, cyclohexenyl, methylcyclohexyl, methylcyclohexenyl, cycloheptyl, cyclooctyl, cyclododecyl or decalinyl radical

or a C₆-C₁₂-aryl or aralkyl radical, in particular a phenyl, benzyl, phenethyl, naphthyl, biphenylyl, anthryl, phenanthryl, azulenyl, anthraquinonyl, 2-, 3- or 4-methylphenyl, 2,3-, 2,4- or 2,5-dimethylphenyl or mesitylyl radical,

or a C₆-C₁₂-heteroaryl or heteroaralkyl radical, in particular a furyl, pyrrolyl, thienyl, benzofuranyl, isobenzofuranyl, benzothiyl, isobenzothienyl, indolyl, isoindolyl, indolizinyl, pyrazolyl, imidazolyl, oxazolyl, thiazolyl, isoxazolyl, isothiazolyl, indazolyl, carbazolyl, benzotriazolyl, purinyl, pyridyl, pyridazinyl, pyrimidyl, pyrazinyl, quinolinyl, isoquinolinyl, quinoxalinyl, quinazolinyl, cinnolinyl, phenanthridinyl, acridinyl, 1,10-phenanthrolinyl, phenazinyl, phenothiazinyl or phenoxazinyl radical,

or is R⁴R⁵R⁶Si where R⁴, R⁵ and R⁶ are each independently C₁-C₁₂-alkyl, in particular methyl, ethyl, n-propyl, isopropyl or n-butyl,

or C₁-C₁₂-oxyalkyl, in particular methoxy, ethoxy, n-propoxy, isopropoxy or butoxy,

C₆-C₁₂-aryl or C₇-C₁₂-aralkyl, in particular phenyl or benzyl,

and R² and R³ are each independently H, C₁-C₁₂-alkyl, in particular methyl, ethyl, n-propyl or n-butyl,

C₆-C₁₂-aryl or C₇-C₁₂-aralkyl, in particular phenyl, 2-, 3- or 4-methylphenyl or benzyl,

and to alkynols R⁷—CO—C≡C—CH₂OH, R⁷—CO—C≡C—CH₂—CH₂OH or R⁷—CO—C≡C—CH₂—CH₂—CH₂OH where R⁷ is methyl, ethyl or a linear or branched C₃-C₁₂ radical, in particular an n-propyl, isopropyl, 1- or 2-n-butyl, 2-methylpropyl, 1-, 2- or 3-n-pentyl, 2- or 3-methyl-1-butyl, 1,2-dimethylpropyl, tert-butyl, neopentyl or tert-pentyl radical,

or a saturated or unsaturated cyclic C₃-C₁₂ radical, in particular a cyclopropyl, cyclobutyl, cyclopentyl, cyclopentenyl, methylcyclopentyl, methylcyclopentenyl, cyclohexyl, cyclohexenyl, methylcyclohexyl, methylcyclohexenyl, cycloheptyl, cyclooctyl, cyclododecyl or decalinyl radical

or a C₆-C₁₂-aryl or aralkyl radical, in particular a phenyl, benzyl, phenethyl, naphthyl, biphenylyl, anthryl, phenanthryl, azulenyl, anthraquinonyl, 2-, 3- or 4-methylphenyl, 2,3-, 2,4- or 2,5-dimethylphenyl or mesitylyl radical,

or a C₆-C₁₂-heteroaryl or heteroaralkyl radical, in particular a furyl, pyrrolyl, thienyl, benzofuranyl, isobenzofuranyl, benzothiyl, isobenzothienyl, indolyl, isoindolyl, indolizinyl, pyrazolyl, imidazolyl, oxazolyl, thiazolyl, isoxazolyl, isothiazolyl, indazolyl, carbazolyl, benzotriazolyl, purinyl, pyridyl, pyridazinyl, pyrimidyl, pyrazinyl, quinolinyl, isoquinolinyl, quinoxalinyl, quinazolinyl, cinnolinyl, phenanthridinyl, acridinyl, 1,10-phenanthrolinyl, phenazinyl, phenothiazinyl or phenoxazinyl radical,

and also to Cl—CH₂—C≡C—CH₂OH and HO—CH₂—C≡C—C≡C—CH₂OH.

Very particular preference is given to 2-propyn-1-ol, but-3-yn-1-ol, but-2-yn-1-ol, pent-4-yn-1,2-diol, 2-butyn-1,4-diol, 4-chloro-2-butyn-1-ol, 4-acetoxy-2-butyn-1-ol, 4-t-butyldimethylsiloxy-2-butyn-1-ol, 3-phenyl-2-propyn-1-ol, 3-trimethylsilyl-2-propyn-1-ol.

In particular, 2-propyn-1-ol, 4-chloro-2-butyn-1-ol and 2-butyn-1,4-diol are suitable.

The nitroxyl compound used as an oxidation catalyst is generally a di-tert-alkylnitroxyl compound.

It is preferably a nitroxyl compound of the general formula I

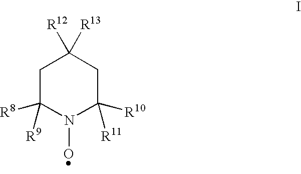

where the radicals R⁸, R⁹, R¹⁰ and R¹¹ are each independently C₁-C₁₂-alkyl or C₂-C₁₂-alkenyl or C₆-C₁₂-aryl or aralkyl,

and the radicals R¹² and R¹³ are each independently hydrogen, OH, CN, halogen,

linear or branched, saturated or unsaturated C₁-C₂₀-alkyl, C₆-C₂₀-aryl, C₆-C₂₀-hetaryl or C₆-C₂₀-aralkyl, OR¹⁴, O—COR¹⁴, O—COOR¹⁴, OCONHR¹⁴, COOH, COR¹⁴, COOR¹⁴, CONHR¹⁴

where R¹⁴ is a linear or branched, saturated or unsaturated C₁-C₂₀-alkyl radical, or a C₆-C₂₀-aryl, C₃-C₂₀-hetaryl or C₆-C₂₀-aralkyl radical,

—(O—CH₂—CH₂)ₙ—OR¹⁵, —(O—C₃H₆)ₙ—OR¹⁵, —(O—(CH₂)₄)ₙ—OR¹⁵, —O—CH₂—CHOH—CH₂—(O—CH₂—CH₂—)ₙ—OR¹⁵

where R¹⁵ is hydrogen, C₁-C₂₀-alkyl, C₆-C₂₀-aralkyl, where n=1 to 100, or CH₂—CHOH—CH₃ or CH₂—CHOH—CH₂—CH₃,

NR¹⁶R¹⁷, NHCOR¹⁶, NHCOOR¹⁶, NHCONHR¹⁶,

where R¹⁶ and R¹⁷ are each independently a linear or branched, saturated or unsaturated C₁-C₂₀-alkyl radical, a C₆-C₁₂-cycloalkyl radical, or a C₆-C₂₀-aryl, C₃-C₂₀-hetaryl or C₆-C₂₀-aralkyl radical,

where the radicals R¹² and R¹³ may also be linked to a ring,

and where the radicals R¹² and R¹³ in turn may also be substituted by COOH, OH, SO₃H, CN, halogen, primary, secondary or tertiary amino or quaternary ammonium,

or the radicals R¹² and R¹³ together may also be ═O, ═NR¹⁸, ═N—OR¹⁸, ═N—N═CR¹⁸R¹⁹ where R¹⁸ and R¹⁹ are each independently hydrogen, C₁-C₂₀-alkyl or C₆-C₂₀-aralkyl.

Preference is further given to the nitroxyl compound being two molecules of the formula I which are linked via a bridge ═N—N═ in the 4-position.

Preference is further given to the nitroxyl compound being two or more molecules of the formula I which are bonded to each other via one of the two radicals R¹² and R¹³. The linking radical is particularly preferably O-alkyl-O, O—CH₂-aryl-CH₂—O, or a bridge of the general formula (O—(CH₂)ₙ—O)ₘ where n=2 to 4 and m=2 to 50, in particular m=2 to 20.

In a further embodiment, the nitroxyl compound is a polymeric structure comprising compounds of the formula I which are linked via the radicals R¹¹ or R¹² or R¹¹ and R¹².

Those skilled in the art are familiar with a variety of such compounds from the prior art (EP 1103537, Cirriminna et al., Chem. Commun. 2000, 1441; Bolm et al., Chem. Commun. 1999, 1795; Bobbitt et al., Chem. Commun. 1996, 2745, Miyazawa and Endo, J. Molec. Catal. 49, 1988, L31; M. J. Verhoef et al. in “Studies in Surface Science and Catalysis”, Vol. 125, p. 465 ff; D. Brunel et al. in “Studies in Surface Science and Catalysis”, Vol. 125, p. 237 ff; Miyazawa and Endo, J. Polymer Sci., Polym. Chem. Ed. 23, 1985, 1527 and 2487; T. Osa, Chem. Lett. 1988, 1423).

In particular, PIPO (polyamine-immobilized piperidinyloxyl), SiO₂-supported TEMPO, polystyrene- and polyacrylic acid-supported TEMPO are particularly suitable.

Particularly preferred nitroxyl compounds are compounds of the general formula I, where R⁸, R⁹, R¹⁰ and R¹¹ are each CH₃

and R¹² and R¹³ are each independently hydrogen, OH, OR¹⁴, O—COR¹⁴, O—COOR¹⁴, OCONHR¹⁴,

where R¹⁴ is a linear or branched, saturated or unsaturated C₁-C₂₀-alkyl radical, or a C₆-C₂₀-aryl or C₆-C₂₀-aralkyl radical,

—(O—CH₂—CH₂)ₙ—OR¹⁵, —(O—C₃H₆)_{n—OR}¹⁵, —(O—(CH₂)₄)ₙ—OR¹⁵, —O—CH₂—CHOH—CH₂—(O—CH₂—CH₂—)ₙ—OR¹⁵

where R¹⁵ is hydrogen, C₁-C₁₀-alkyl or C₆-C₁₀-aralkyl, where n=1 to 100, or CH₂—CHOH—CH₃ or CH₂—CHOH—CH₂—CH₃,

NR¹⁶R¹⁷, NHCOR¹⁷, NHCOOR¹⁷, NHCONHR¹⁷,

where R₁₆ and R¹⁷ are each independently hydrogen, a linear or branched, saturated or unsaturated C₁-C₂₀-alkyl radical, a C₆-C₁₂-cycloalkyl radical or a C₆-C₂₀-aryl or C₆-C₂₀-aralkyl radical.

Further particularly preferred nitroxyl compounds are compounds of the general formula I where R⁸, R⁹, R¹⁰ and R¹¹ are each CH₃

where R¹² and R¹³ together form ketal groups of the formulae O—CHR²⁰CHR²¹—O or O—CH₂CR²²R²³—CH₂—O where R²⁰, R²¹, R²² and R²³ are each independently hydrogen or C₁-C₃-alkyl,

or where the radicals R¹² and R¹³ together are ═O.

A preferred nitroxyl compound is in particular a compound of the general formula I where R⁸, R⁹, R¹⁰ and R¹¹ are each CH₃

where R¹² is hydrogen and R¹³ is hydrogen, OH, OR¹⁴, O—COR¹⁴,

where R¹⁴ is a linear or branched saturated C₁-C₁₂-alkyl radical, or is an aryl or benzyl radical,

—(O—CH₂—CH₂)ₙ—OR¹⁵, —(O—C₃H₆)ₙ—OR¹⁵, —(O—(CH₂)₄)ₙ—OR¹⁵, —O—CH₂—CHOH—CH₂—(O—CH₂—CH₂—)ₙ—OR¹⁵ where n=1 to 50

and R¹⁵ is hydrogen or CH₂—CHOH—CH₃ or CH₂—CHOH—CH₂—CH₃

NR¹⁶R¹⁷, NHCOR¹⁷ where R¹⁶ and R¹⁷ are each independently a linear or branched, saturated C₁-C₁₂-alkyl radical or an aryl or benzyl radical.

Examples of nitroxyl compounds which can be used with particular preference are TEMPO, 4-hydroxy-TEMPO, 4-oxo-TEMPO, 4-amino-TEMPO, 4-acetamido-TEMPO, 4-benzoyloxy-TEMPO, 4-acetoxy-TEMPO and PIPO.

The nitroxyl compound is generally used in amounts of from 0.01 to 50 mol %, preferably in amounts of from 0.1 to 20 mol %, more preferably in amounts of from 1 to 10 mol %, based on the amount of alkyne alcohol to be oxidized.

The nitroxyl compound may be dissolved in the reaction component comprising the alkyne alcohol or in the aqueous phase or used in supported form as an independent phase.

Those skilled in the art are familiar with suitable hypohalites and hypohalite preparations from the prior art (Ullmann Encyclopedia, 6th Edition, 2001 electronic release; “Chlorine oxides and Chlorine oxygen acids 2.-4.”).

The oxidant used is preferably a compound selected from the group of the hypohalites, in particular hypochlorite, hypobromite and hypoiodite or their mixtures. A particularly preferred oxidant is hypochlorite. Preferred counterions are hydrogen, sodium, potassium, calcium or tetraalkylammonium.

In a particularly preferred embodiment, technical hypohalite solutions, in particular technical hypochlorite solutions, are used.

The oxidant used may also be generated in situ, in particular electrochemically, by hydrolysis, in particular by hydrolysis of N-chlorine compounds, or by redox reactions such as, in the case of hypochlorite or hypobromite solutions, by the disproportionation of chlorine or bromine in aqueous alkaline solution, or by the redox reaction between hypochlorite and bromide which leads to the formation of hypobromite.

The oxidants used, in particular hypochlorite, hypobromite and hypoiodite are preferably used as aqueous solutions in concentrations of from 0.1 M up to their respective saturation concentration.

The pH of the aqueous solutions of the oxidant is generally from 7 to 14, preferably from 7 to 11, more preferably from pH 8 to 10. The desired pH is generally set by adding an acid, preferably sulfuric acid, carbon dioxide, acetic acid, formic acid, hydrochloric acid or phosphoric acid, more preferably carbon dioxide or acetic acid, or a base, preferably sodium hydroxide and potassium hydroxide, more preferably sodium hydroxide. The pH may also be set by adding a buffer, preferably sodium hydrogencarbonate or sodium dihydrogenphosphate, more preferably sodium hydrogencarbonate.

Further possible additives include salts, for example alkali metal, alkaline earth metal or ammonium halides, carbonates or sulfates.

The pH of the aqueous phase of the reaction mixture is generally less than 7, preferably from 1 to less than 7, more preferably from 3 to 6.

The desired pH of the reaction mixture is generally set by adding an acid, preferably sulfuric acid, carbon dioxide, acetic acid, formic acid, hydrochloric acid or phosphoric acid, more preferably sulfuric acid, hydrochloric acid, acetic acid or phosphoric acid, or a base, preferably sodium hydroxide, potassium hydroxide, sodium carbonate or sodium hydrogencarbonate, more preferably sodium hydroxide. The pH may also be set by adding a buffer, preferably sodium hydrogencarbonate or sodium dihydrogenphosphate, more preferably sodium hydrogencarbonate.

When preparing carboxylic acids with high acidity, for example propiolic acid or acetylenedicarboxylic acid, the pH of the reaction mixture is held below 7 by the acid being formed.

The reaction temperature is generally from −10 to +80° C., preferably from −5 to +30° C., more preferably from −5 to +15° C.

The processes are preferably carried out at atmospheric pressure.

All of the above symbols of the above formulae are each defined independently of one another.

### DETAILED DESCRIPTION OF PREFERRED EMBODIMENTS

Other objects and features of the present invention will become apparent from the following detailed description considered in connection with the accompanying examples which disclose several embodiments of the present invention. It should be understood, however, that the examples are designed for the purpose of illustration only and not as a definition of the limits of the invention.

### EXAMPLES

### Comparative Example 1

### Oxidation of 2-propyn-1-ol at pH>7

7.5 g (134 mmol) of 2-propyn-1-ol together with 1.2 g (7 mmol) of 4-hydroxy-TEMPO are dissolved in 10 ml of water and cooled to 5° C.

800 g of 2.2 M sodium hypochlorite solution (technical bleaching liquor; set in advance to pH 9.5 with gaseous CO₂) were added with intensive stirring at such a rate that the internal temperature did not rise above 10° C. The pH of the reaction mixture is maintained at >8 by adding sodium hydroxide solution.

After completed addition, stirring is continued at max. 10° C. for a further 1 hour. Analysis of the reaction mixture shows that about 40% of the alcohol used were not converted and the reaction mixture contains virtually no propiolic acid.

### Example 1

### Oxidation of 2-propyn-1-ol to Propiolic Acid

75 g (1340 mmol) of 2-propyn-1-ol together with 11.5 g (67 mmol) of 4-hydroxy-TEMPO are dissolved in 75 ml of water and cooled to 5° C.

1563 g of 2.2 M sodium hypochlorite solution (about 1.2 l of technical bleaching liquor; set in advance to pH 9.5 using gaseous CO₂) were added with intensive stirring at such a rate that the internal temperature did not rise above 10° C. and, at the same time, the pH did not rise above 7. The first 20% in particular of the bleaching liquor therefore have to be added slowly.

After completed addition, extraction is effected using a total of 600 ml of methyl t-butyl ether (MTBE) (this results in relatively small amounts of propargyl propiolate). After the removal of the organic phases, the aqueous phase is set to pH 0 using about 64 g of concentrated sulfuric acid and extracted 3 times with 300 ml of MTBE each time. The aqueous phase is disposed of.

The MTBE phases are combined, dried and, after partial distillative removal of the MTBE, provide an about 50% solution of propiolic acid in MTBE which contains 41 g (590 mmol) of propiolic acid corresponding to a 44% yield and having a purity of 90-92%. This solution may be used directly for synthesis of, for example, propiolic esters.

### Example 2

### Continuous Oxidation of 2-propyn-1-ol to Propiolic Acid

112 g (2.0 mol) of 2-propyn-1-ol together with 17.2 g (100 mmol) of 4-hydroxy-TEMPO are dissolved in 112 ml of water (solution A). 3 kg of 2.2 M sodium hypochlorite solution (technical bleaching liquor) are set to pH 9.5 using acetic acid (solution B). Solution A at 1.11 g/min (corresponding to 9.3 mmol of propargyl alcohol/min) and solution B at 13.9 g/min (corresponding to 25 mmol of hypochlorite/min) are conveyed with intensive stirring into a 1 liter stirred tank from which the reaction mixture is removed continuously at the same time at such a rate that the volume of the reaction mixture remains constant at about 600 ml (corresponding to an average residence time of about 40 min). The temperature is maintained between 5 and 10° C. by cooling. The pH of the reaction mixture is between 5 and 6.

The continuously withdrawn reaction mixture is washed with MTBE in a similar manner to example 3 and set to pH 0 using sulfuric acid, and the reaction product is isolated from the aqueous phase using MTBE. The yields of propiolic acid obtained in this way are 50-60%.

### Example 3

### Biphasic Oxidation of 2-propyn-1-ol to Propargyl Propiolate or Propiolic Acid

A solution of 10 g (180 mmol) of 2-propyn-1-ol in 30 ml of methylene chloride is admixed with 1.55 g (9 mmol) of 4-hydroxy-TEMPO and cooled to 5° C.

207 g of 2.2 M sodium hypochlorite solution (technical bleaching liquor; set in advance to pH 9.5 using gaseous CO₂) are added with intensive stirring at such a rate that the internal temperature does not rise above 10° C. and the pH does not rise above 7.

After completed addition, the pH is 3.3. The organic and aqueous acid phases are separated. The organic phase contains 1.7 g of propargyl propiolate. This is admixed with stirring with 50 ml of 1 N NaOH at 35° C. and removed after 1 h. The alkaline aqueous phase is repeatedly extracted using methyl t-butyl ether (MTBE). The combined MTBE phases contain propargyl alcohol which is unconverted or has been recovered from the ester hydrolysis and can be reused.

The aqueous acidic and alkaline phases are likewise combined, set to pH 0 using concentrated sulfuric acid and extracted 3 times with 100 ml of MTBE each time. The aqueous phase is disposed of.

The MTBE phases are combined, dried and, after partial distillative removal of the MTBE, provide a 50% solution of propiolic acid in MTBE which contains 9.0 g (129 mmol) of propiolic acid, corresponding to a 71% yield. This solution may be used directly for synthesis of, for example, propiolic esters.

### Example 4

### Oxidation of 3-butyn-1-ol to Acetylene Acetic Acid

91 g (1.3 mol) of 3-butyn-1-ol together with 11.5 g (67 mmol) of 4-hydroxy-TEMPO are dissolved in 75 ml of water and cooled to 5° C.

1563 g of 2.2 M sodium hypochlorite solution (technical bleaching liquor; set in advance to pH 9.5 using gaseous CO₂) are added with intensive stirring at such a rate that the internal temperature does not rise above 10° C. and at the same time the pH does not rise above 7. After completed addition, the pH is set to 7 using sodium hydroxide solution and extraction is effected using a total of 500 ml of methyl t-butyl ether (MTBE). After removing the organic phases, the aqueous phase is set to pH 1 using concentrated sulfuric acid and extraction is effected twice with 300 ml of MTBE each time. The aqueous phase is disposed of.

The MTBE phases are combined, dried and, after distillative removal of the MTBE, provide acetyleneacetic acid in 41% yield.

### Example 5

### Oxidation of 2-butyn-1,4-diol to Acetylenedicarboxylic Acid

A solution of 4.76 g (55.3 mmol) of 2-butyn-1,4-diol in 50 ml of water is admixed with 0.63 g (3.66 mmol) of 4-hydroxy-TEMPO, the mixture is cooled to 3° C. and admixed with stirring and cooling to a max. temp. of 10° C. with 111 ml of 1.8 M sodium hypochlorite solution (technical bleaching liquor; set in advance to pH 9.5 using gaseous CO₂) within 1 hour. Reaction is allowed to continue for a further 2 hours, then the mixture is shaken with 50 ml of MTBE, the aqueous phase is acidified to pH 0 using concentrated sulfuric acid and the water phase is extracted twice with 50 ml of MTBE each time. Concentration of the two MTBE extracts by evaporation under reduced pressure provides 6.3 g (50%) of acetylenedicarboxylic acid in the form of colorless crystals.

Accordingly, while a few embodiments of the present invention have been shown and described, it is to be understood that many changes and modifications may be made thereunto without departing from the spirit and scope of the invention as defined in the appended claims.

What is claimed is:

1. A process for preparing alkynecarboxylic acids, comprising
an oxidation reaction of an alkyne alcohol
with from 1 to 10 molar equivalents of a hypohalite based on the number of functional groups to be oxidized
in the presence of a nitroxyl compound
at a pH of less than 7.

2. A process for preparing alkyne alcohol esters of alkynecarboxylic acids, comprising
a partial oxidation reaction of an alkyne alcohol to alkyne alcohol esters of alkynecarboxylic acids
with from 0.5 to 5 molar equivalents of a hypohalite based on the number of functional groups to be oxidized
in the presence of a nitroxyl compound
at a pH of less than 7.

3. The process as claimed in claim 1, wherein an aqueous phase is used.

4. The process as claimed in claim 3, wherein the aqueous phase comprises at least one water-miscible solvent.

5. The process as claimed in claim 1, wherein the reaction is carried out in a multiphasic system.

6. The process as claimed in claim 5, wherein at least one aqueous phase and at least one organic phase are used.

7. The process as claimed in claim 1, wherein the reaction is carried out continuously.

8. The process as claimed in claim 3, wherein the aqueous phase has a reaction mixture with a pH from 3 to 6.

9. The process as claimed in claim 2, wherein an aqueous phase is used.

10. The process as claimed in claim 9, wherein the aqueous phase comprises at least one water-miscible solvent.

11. The process as claimed in claim 2, wherein the reaction is carried out in a multiphasic system.

12. The process as claimed in claim 11, wherein at least one aqueous phase and at least one organic phase are used.

13. The process as claimed in claim 2, wherein the reaction is carried out continuously.

14. The process as claimed in claim 9, wherein the aqueous phase has a reaction mixture with a pH from 3 to 6.
